(19) United States
(12) Patent Application Publication
(10) Pub. No.: US 2009/0088625 A1
(43) Pub. Date: Apr. 2, 2009

(54) Photonic Based Non-Invasive Surgery System That Includes Automated Cell Control and Eradication Via Pre-Calculated Feed-Forward Control Plus Image Feedback Control For Targeted Energy Delivery

(76) Inventors: Kenneth Oosting, Rocklin, CA (US); Amy Snook, Rocklin, CA (US)

Correspondence Address: Jones Day;Attention: Peter G. Thurlow, 222 East 41st Street, New York, NY, 10017, US

(21) Appl. No.: 12/242,577

(22) Filed: Sep. 30, 2008

Related U.S. Application Data
(60) Provisional application No. 60/976,699, filed on Oct. 1, 2007.
(60) Provisional application No. 60/982,542, filed on Oct. 25, 2007.
(60) Provisional application No. 61/021,941, filed on Jan. 18, 2008.

(51) Int. Cl. A61B 5/055; A61B 5/05

(52) U.S. Cl. 600/411; 600/425

(57) ABSTRACT

A photonic based non-invasive surgery system that includes an imaging device such as an MRI device and at least two beam generators for generating beams of energy for delivery to a target in the person's body, where the beams of energy intersect at a point. The system also includes a feed-forward control for precalculating anticipated deflections and resulting pathways as the beams of energy travel throughout the person's body, and a feedback control to obtain and use information gathered by the imaging device.

### CROSS-REFERENCE TO RELATED APPLICATIONS

This application claims priority to provisional application Nos. 60/976,699, which was filed in the U.S. Patent and Trademark Office (“USPTO”) on Oct. 1, 2007; 60/982,542, which was filed in the USPTO on Oct. 25, 2007; and 61/021,941, which was filed in the USPTO on Jan. 18, 2008; and incorporates by reference the information in provisional application No. 60/954,364, filed on Aug. 7, 2007.

### STATEMENT REGARDING FEDERALLY SPONSORED RESEARCH OR DEVELOPMENT

This application is the subject of a grant application request, filed on Aug. 5, 2008, in the National Institutes of Health under grant no. 00499945 having a CFDA tracking number 93.394. The information included in the grant request is incorporated herein by reference.

### THE NAMES OF THE PARTIES TO A JOINT RESEARCH AGREEMENT

There has been no joint research agreements entered into with any third-parties.

### BACKGROUND OF THE EMBODIMENTS OF THE PRESENT INVENTION

Cancer treatment systems that use an MRI device and a beam generator are known in the art. A number of existing treatment systems damage healthy tissue surrounding the cancerous tissue being treated. The systems described in this application improve existing cancer treatment systems to, among other things, minimize damage to the healthy tissue in the area surrounding the cancerous tissue being treated and provides greater assurance that target tissue is killed.

### BRIEF SUMMARY OF THE EMBODIMENTS OF THE PRESENT INVENTION

An embodiment of the present invention is directed to a photonic based non-invasive surgery system that includes an imaging device for taking an image of a person's body to provide details of internal physiology, and at least two beam generators for generating beams of energy for delivery to a target in the person's body, where the beams of energy intersect at a point. The system also includes a means for feed-forward control for precalculating anticipated deflections and resulting pathways as the beams of energy travel throughout the person's body, and a means for feedback control through information gathered by the imaging device, where the means for feed-forward control and the means for feedback control function in an integrated manner.

Another embodiment of the present invention is directed to a photonic based non-invasive surgery system that includes an imaging device for taking an image of a person's body to provide details of internal physiology, and at least two beam generators for generating beams of energy for delivery to a target in the person's body along a certain pathway, where the beams of energy intersect at a certain point and where the beams of energy include different types of energy for delivery to the target along the certain pathway. The system also includes a means for feed-forward control for precalculating anticipated deflections and the certain pathway as the beams of energy travel throughout the person's body, and a means for feedback control through information gathered by the imaging device.

Yet another embodiment of the present invention is directed to a photonic based non-invasive surgery system. The system includes an imaging device for taking an image of a person's body to provide details of internal physiology and at least two beam generators for generating beams of energy for delivery to a target in the person's body, where the beams of energy intersect at a certain point. The system includes a means for feed-forward control for precalculating anticipated deflections and resulting pathway as the beams of energy travel throughout the person's body and a means for feedback control through information gathered by the imaging device and a plurality of nanoparticles attached to the target or within the target.

Another embodiment of the present invention is directed to a photonic based non-invasive surgery system. The system includes an imaging device for taking an image of a person's body to provide details of internal physiology and at least one beam generator for generating beams of energy for delivery to a target in the person's body. At least one beam generator includes a beam processor for processing the beam from the beam generator and the beams of energy intersect at a certain point. The system also includes a means for feed-forward control for precalculating anticipated deflections and resulting pathway as the beams of energy travel throughout the person's body, a means for feedback control through information gathered by the imaging device and a plurality of nanoparticles attached to the target or within the target.

### BRIEF DESCRIPTION OF THE SEVERAL VIEWS OF THE DRAWINGS

FIG. 1A is a view from the forward end of the MRI device.

FIG. 1B is a view from the side of the MRI device.

FIG. 2 is a system block diagram showing the features in an embodiment of the present invention.

FIG. 3A is a view showing a beam generator and the beam's deflection when the beam comes into contact with the skin's surface.

FIG. 3B is an enhanced view showing the beam's angle of incidence, angle of deflection and angle of dispersion.

FIG. 4 is a view showing a beam generator and the beam's deflection when the beam comes into contact with a person's skin, bones and tendons before the beam reaches the target cells.

FIG. 5 is a view showing a scale and various parameters including watts, gradient, absorption and cell death when one to four beams of energy are used in embodiments of the present invention.

FIG. 6 is a view showing four electromagnetic beams in three dimensional space.

FIGS. 7A-7D shows the intersection point with three cylinders (FIGS. 7A and 7B) and six cylinders (FIGS. 7C and 7D).

FIGS. 8A and 8B are views showing electromagnetic waves, two waves in phase (FIG. 8A) and two waves out of phase (FIG. 8B).

FIGS. 9A-9D are views showing the intersection point with two beams (FIGS. 9A-9C) and three beams (FIG. 9D).

FIG. 10 is a view of an electromagnetic wave.

FIG. 11 shows three effectiveness curves when an embodiment of the present invention uses three beams and nanoparticles are attached to the organelle within cells (curve A), when an embodiment of the present invention uses a single beam and nanoparticles attached to an organelle within cells (curve B) and when conventional radiation is used (curve C).

FIG. 12 shows a two story embodiment of the present invention having an MRI or CT image scanner located on a first level and three beam generators disposed on a lower level.

FIG. 13 shows an x-ray beam used in combination with a beam generation unit.

FIGS. 14-19 show a sequence of six frames of a control sequence for an embodiment of the present invention that takes into consideration errors in the initial aiming of the beam generator in relation to the target, uses the feedback error values in conjunction with the feed-forward control to adjust the beam until the beam converges on the target area and after releasing the beam pulse, to destroy the target cell.

FIG. 20 shows an embodiment of a beam splitter and aimer (beam processor) where the mirror shield and tunnel, the first mirror and the waveguide cluster are the components of the beam splitter and the final mirror is the aimer.

### DETAILED DESCRIPTION OF THE EMBODIMENTS OF THE INVENTION

The embodiments of the present invention as described in this application provide a system for targeting specific cells such as cancer cells or groups of cells including cancerous and non-cancerous cells for the delivery of energy such as radiation. These embodiments also describe the integrated method and delivery system that is capable of delivering energy to a specific cell or group of cells with minimal or no damage to the surrounding tissue.

The technology as described in this patent application is an innovation in itself but it also involves the integration of many other technologies such as imaging, radiation, microwave, ultrasound, lasers, robotics, and more. The embodiments of the present invention include subject matter directed to targeting, control, energy delivery strategy, energy delivery mechanics, and systems integration.

The benefits of the technology are far reaching and extend even beyond healthcare. However, healthcare applications are the initial focus of the invention. For example, the invention as described herein may be used to eradicate cancer cells anywhere in the body without the need for surgery. Elimination of cancer cells applies to tumors and may also apply to metastasized cancers that have spread throughout the body. As the technology is developed it may be capable of eliminating viruses and bacterial infections from the body. Diseases such as Hepatitis B and AIDS may be cured. Other potential applications include selective elimination of cells in the prostate or other parts of the body. Reducing the size of the organ or improving its function or destruction of fat cells for health or cosmetic reasons are also potential applications. Non-cell material may also be destroyed or loosened for benefits such as improving blood flow or making joints move more freely.

Materials Science—Analysis, Testing and Repair. The field of material science makes use of x-rays to analyze, test and repair materials. The availability of the present invention's high energy intersection point and accurate aiming could be of significant value in this industry. The present invention will provide the capability to pinpoint problems and take action to correct problems on a microscopic level.

Laboratory Uses—Chemical Analysis and Crystal Analysis. Scientist working on the analysis of chemical compounds and crystals will find the present invention's technology useful to expedite research projects and collect data that might otherwise be illusive. The size and accurate control of the present invention's intersection point would again be the anticipated benefit as compared to other alternatives for this type of work.

Eradication of harmful or unwanted cells can improve the function of surrounding cells. Thus, the methods and systems described herein can be used to control or improve the function of cells. In some applications, energy of a lower intensity may be delivered to the target point to stimulate cells or provide cell therapy. Cell therapy may also include thinning the cell membrane, moving cells, disintegrating or destroying unwanted internal cellular material, disintegrating or destroying unwanted external or non-cellular material, and stimulating internal organelles through use of harmonics.

The Present Invention vs. Radiotherapy: Even when x-ray energy is used, the present invention differs from radiotherapy in five substantial ways. First, it uses a different modality to cause cell death. Second, it is dependent on volume of photons per second to increase wattage rather than energy per photon. Third, it makes better use of the cell's own devices to cause immediate cell death in a single treatment. Fourth, the present invention avoids most DNA damage by using less total energy and less energy per photon. Fifth, the target selectivity mechanism of the present invention is mechanical, external and controllable compared to the target selectivity of radiotherapy which is biological, internal, uncontrollable, and uncertain.

Radiotherapy seeks to create large numbers of free radicals which in turn cause double strand breaks in the DNA double helix strands. The body has a built in repair mechanism for this type of cell damage. Therefore, radiotherapy must overwhelm the repair mechanism with a large volume of these breaks. Each treatment causes the body to increase its efforts in repair, making subsequent treatments less and less effective.

The present invention causes the immediate (within the few milliseconds of treatment for a given cell) disruption of the processes within the cell and ruptures internal membranes of the cell to cause cell death. These disruptions and ruptures directly initiate apoptosis or self-digestion of the cell. Apoptosis will also eventually occur with radiotherapy in cells that die. However, there is only a statistical probability that any given cell will die and cell death might take days or even weeks after treatment. This is because the interactions in radiotherapy are much farther up the chain of events from apoptosis than the interactions in the present invention. The farther up the chain of events the less certain the results.

The present invention uses a plurality of beams to increase the wattage to a small target volume. Each beam introduces additional energy into the intersection point. The sum of the electron voltage (energy) in the intersection point is controlled by the number of beams as opposed to the greater electron voltage of the individual photons in radiotherapy. This difference is important in that it allows the present invention to: a) use photon energies that are more readily absorbed, and b) introduce less total energy into the person.

The peak energy arriving at the target is similar in absolute value between the present invention and radiotherapy but it is in a different form. The form used in the present invention is a comparatively high number of lower energy photons as opposed to radiotherapy's use of a lower number of higher energy photons.

The present invention uses high wattage in the intersection point to create chemical and physiological disruptions sufficient to rupture the membranes of cell organelles without burning the cell. These disruptions are the result of a large volume of photon attenuations in low energy interactions. Radiotherapy is more dependent on the higher energy range Compton interactions with high production of free radicals capable of breaking DNA strands.

The present invention uses its intersection point and/or nano technology to assure selectivity of target cells as opposed to non-target cells. The computers and manipulators in the present invention's architecture control the selectivity associated with the intersection point. Monoclonal antibodies or other targeting molecules attached to nanoparticles provide a second degree of selectivity for the present invention. The targeting molecules cause the nanoparticles to accumulate in much higher concentrations in cells of a certain type. Cancer cells can be targeted in this way for instance. Because the nanoparticles substantially reduce the amount of energy required to allow the present invention to cause cell death, the output energy of the present invention can be adjusted down to a level such that no cell death occurs except when the beams intersect where the concentration of nanoparticles is above the threshold that identifies a cell as a target. In this way, the present invention doubles its targeting assurance; the failure mode is that nothing happens to a cell if either the nanoparticle concentration is too low or the intersection point does not hit the cell. This degree of selectivity is critical when working close to nerves or other sensitive tissue.

Radiotherapy is dependent upon the cell division cycle to select which cells will be destroyed. The cells are particularly vulnerable to DNA strand breaks at certain stages of cellular reproduction. Cancer cells spend much more time in reproduction and are therefore more vulnerable to radiotherapy. However, there is only a higher probability the cancer cells will be in this state. In reality, some normal cells will be in division and will be killed and some cancer cells will not be dividing at the time of radiotherapy treatment and will be relatively immune to the treatment. This is why radiotherapy takes multiple treatments and often fails to kill all of the cancer. There is no way to be certain that the target cells in any volume will be killed during radiotherapy. The corollary is also true: there is no way to be certain that the healthy cells in the path of the radiotherapy beam will not be killed.

The present invention also has the potential to kill healthy cells but the probability of such is substantially lower than with radiotherapy. In fact, the present invention is no more likely to cause such damage than a diagnostic x-ray (the present invention may even be less likely).

The risk of secondary cancers is also much higher with radiotherapy than with the present invention. This is due to the difference in total energy introduced in the patient as well as the difference in primary cell death modalities. Intended breaks in DNA strands can fail and result in modifications to DNA strands. Some percentage of these modifications become secondary cancers.

Applicant notes that the embodiments of the present invention are directed to treating human beings; however, animals such as dogs, cats, etc. may also be treated using the present invention and are included in the definition of “person” as used in the claims.

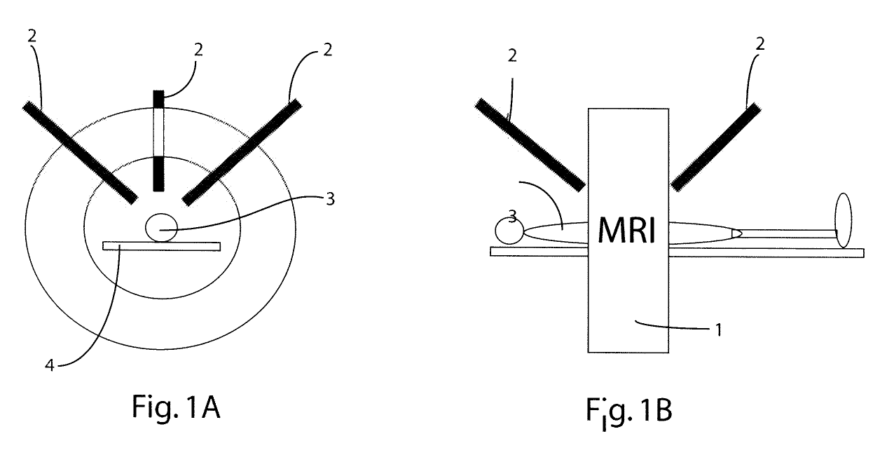

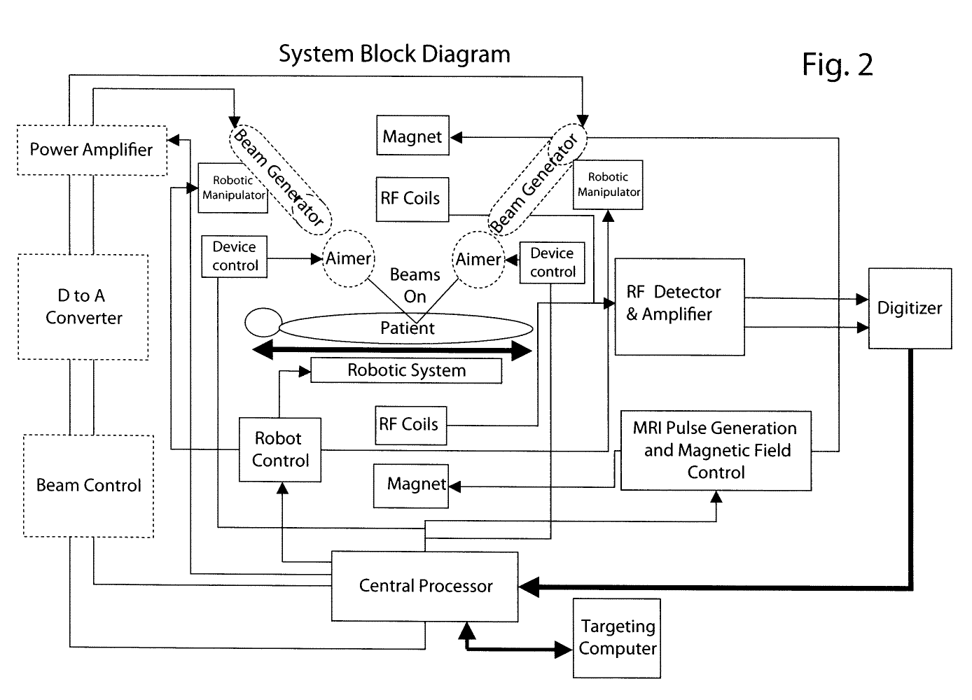

As shown in FIGS. 1A and 1B, the embodiments of the present invention use existing image acquisition systems such as magnetic resonance imaging (MRI) 1 or computed tomography (“CT” or CAT Scan) to output image information into a control system shown in FIG. 2 which targets certain cells for destruction. The control system then aims two or more preferably very narrow beams 2 at the target area in the person 3 using continuous feedback from the imaging system to verify and refine its aiming. As described in further detail below, the system controls the beams and their intensity such that when the beams converge on the target a burst of intensity is released. In the particular embodiment shown in FIG. 1A, a view of the person 3 resting on a movable horizontal platform 4 is shown. This embodiment shows three orthogonal beam generators 2 disposed above the person and FIG. 1B shows another embodiment from a side of the person 3 that includes two orthogonal beam generators. A person of ordinary skill in the art would readily understand that the beam generators 2 may be disposed under or on the side of the MRI device 1.

The number of beams 2 used is two or more depending on the application. Each beam's maximum energy delivery is less than the minimum to cause damage to cells. However, at a focal point where the beams cross, the energy level is 2, 3, 4 . . . times greater depending on the number of beams used. Such an application allows the device to destroy cells deep within the body without damage to the surrounding tissue. Each beam passing through the body (including beams going to and from the target area) has sufficiently low energy to prevent and/or minimize the damage to healthy tissue surrounding a target area, i.e., only those cells in the point of intersection receive sufficient energy to be destroyed.

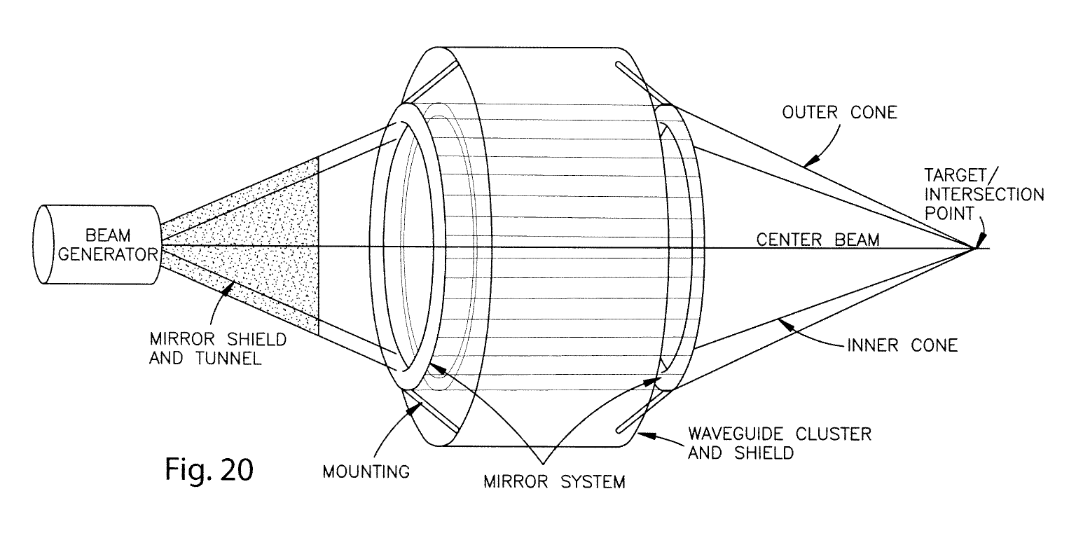

Beam Splitter Concept: As shown in FIG. 20, this embodiment is an alternative to using totally separate beam generators. A single beam generator (x-ray tube, linac, . . . ) can be divided into multiple beam elements. These multiple beam elements can then be deflected and used to create an intersection point. This reduces the cost and complexity by eliminating beam generators but adds some cost and complexity for processing the beam elements of the one remaining beam generator. This embodiment of the present invention uses x-ray mirrors to select and direct beam elements to drive through multiple wave guides and then use additional mirrors to direct each beam element to an intersection point. In addition, another embodiment of the present invention includes each of the multiple beam generators having a beam processor (the beam processor includes a beam splitter and aiming device) associated with it. An embodiment having the features of a beam processor associated with each of the beam generators will increase the wattage in the intersection point and expand the usefulness of the present invention.

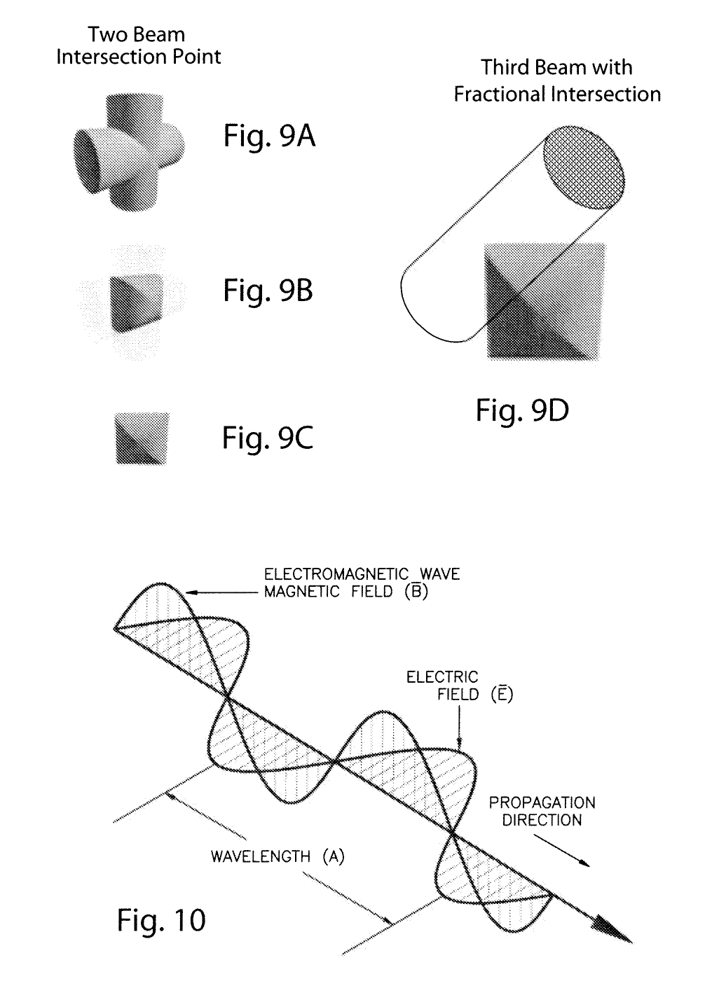

As shown in FIGS. 9A-9D, the intersections of beams provide smaller intersection points for finer work. The embodiment shown in FIGS. 9A-9C shows the intersection point of two beams while the embodiment shown in FIG. 9D shows a third beam with fractional intersection combined with the two beams shown in FIG. 9A. This requires even greater precision in the aiming of the individual beams and is accomplished via additional signal processing to provide an “energy level” feedback loop whereby the electromagnetic energy within the intersection point is measured. This energy level is proportional to the percentage or fraction of the intersection for beams of constant power. The size of the intersection point may then be measured as a function of the energy level feedback.

The systems described herein would, among other things, target and rupture the mitochondria, lysosomes or other organelles within a cell, which would result in the cell being dissolved from within. A person of ordinary skill in the art would readily understand that there may be multiple mitochondria in some cells, and that there are multiple lysosomes and other organelles in each cell. Killing the cell would require the majority to be ruptured. Cell death by any mechanism would result in the eventual digestion of the cell. Attacking the mitochondria, lysosomes or other organelles that will trigger digestion of the cell provides the significant benefit of reducing the amount of energy required to accomplish the task of killing the given cell.

A preferred method of accomplishing cell death is the use of nanoparticles that attach to an organelle within cells. The types of nanoparticles include gold, carbon, iron, magnetic material, compound metal, tubes, balls, bubbles, springs, coils, rods and combinations thereof. Thus, the molecular heating within the organelle causes expansion and rupture or heating of the cell that leads to the cell's death. Another approach of accomplishing cell death is to use interlaced harmonics in the beam stream to affect cellular material.

Alternatively, the outer membrane of the cell may be ruptured to kill the cell. This may be done using fractional intersection of beams as shown in FIG. 9D such that a small portion of the cell membrane is inside the intersection point. The burst of energy at the focal point caused by the intersection of the beams creates a hot spot resulting in a hole in the cell membrane. The hole allows the escape of cellular materials and death of the cell. This method kills two or more cells at a time as the membrane of adjacent cells are also ruptured. The systems described herein will destroy single cells or small groups of cells using one or all of the above methods. The limiting factors are image resolution, beam size, targeting, and aiming.

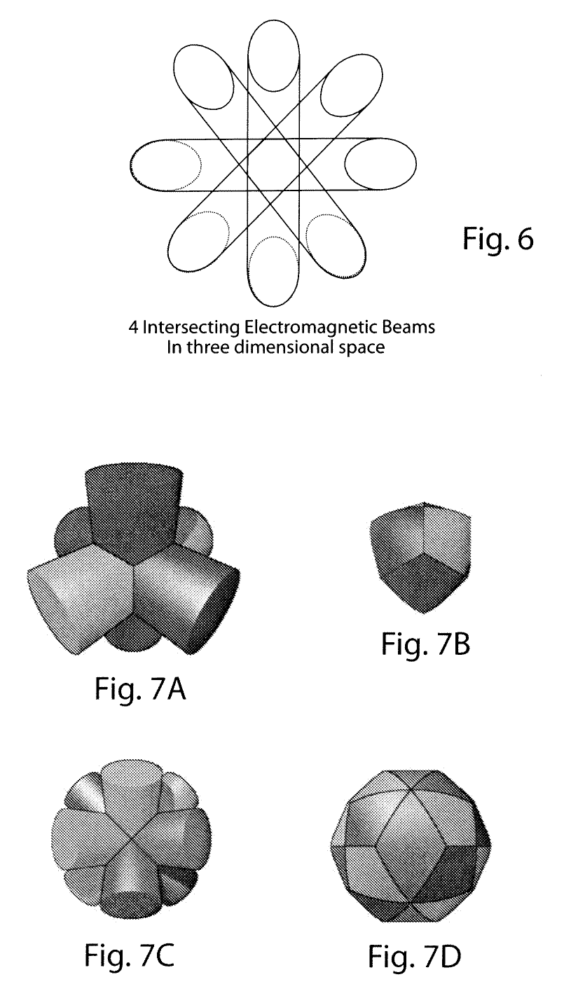

As described above, a preferred embodiment of the present invention includes using a plurality of beams that individually do not adversely affect healthy tissue surrounding a target area as they pass throughout a person's body. However, when the plurality of beams intersect a burst of energy is created that kills the targeted cells. FIGS. 6 and 7A-D help a person of ordinary skill in the art to further understand the dynamics directed to the intersection point. FIG. 6, for example, shows four intersecting electromagnetic beams in a three dimensional space. Because the beams are electromagnetic, there is no need for them to be coplanar. In fact, the waves do not even have to be phase aligned if they are orthogonal. The amplitude of the intersecting waves is the sum of the amplitudes of the individual waves which would be the maximum kiloelectron volts (KeV) at multiple points within the intersection point. Each repeating wave element would display this maximum KeV.

A person of ordinary skill in the art would readily understand that technically, a focal point is the small, three-dimensional volume being targeted by the preferred embodiments of the present invention described herein. In general a point has no dimensions. The focal point is defined by the intersection of the beams and is approximately the size of the sphere created by the spinning cross section of the beams given that the beams are cylindrical and of generally equal size. As shown in FIGS. 7A-D, the actual shape of the intersection point is called a Steinmetz solid. FIG. 7A shows, for example, a rhombic dodecahedron having three cylinders where the cylinders travel through the center of each face. Such an arrangement is the same as cylinders that travel through the vertices of an octahedron. Moreover, in this arrangement, the volume of such a 3-cylinder rhombic dodecahedron is determined by the formula (16−sqrt(128))r3. FIG. 7C shows, for example, a cube-octahedron having six cylinders through the midpoint of each edge. Moreover, in this arrangement, the volume of such a cube-octahedron is (16/3)(3=sqrt(12)−sqrt(32))r3. When using a preferred embodiment of the present invention, orthogonal beams provide the preferred and best possible targeting, as their intersection point is the minimum size. For beams of different sizes or for non-orthogonal beams, the intersection point has the shape of a modified Steinmetz solid (not shown). For applications requiring more than three beams, the size and shape of the intersection point of the beams is less dependent on the angle of convergence.

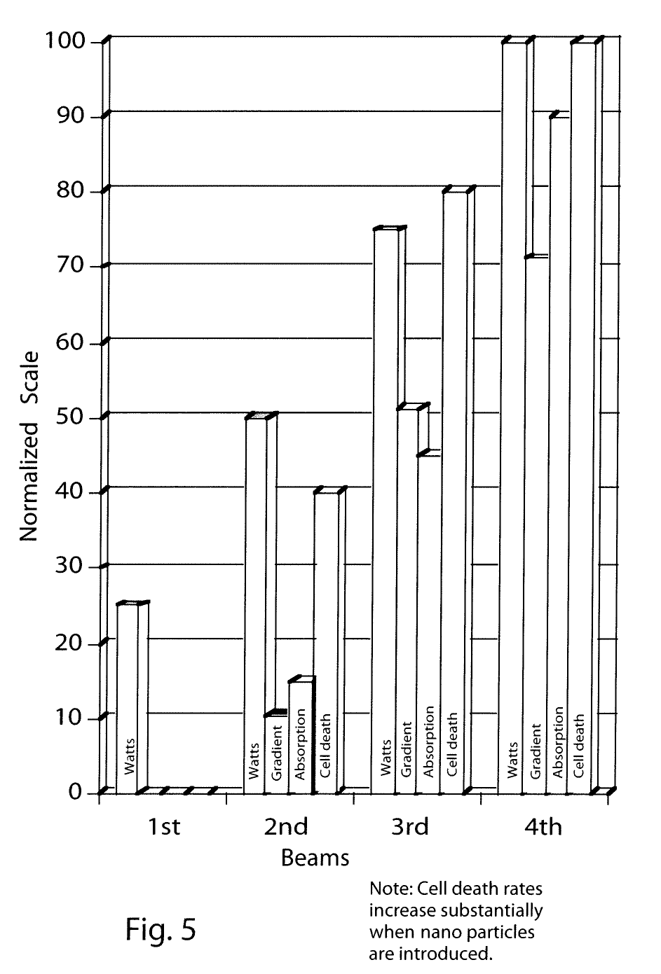

FIG. 5 also shows that the lower concentration modes of the beams improve image resolution and control function. In particular, FIG. 5 shows the general expected results of using multiple beams of ionizing radiation at the focal point. For example, the watts, gradient, cell absorption and cell death are greatest when four beams are used. Like an electron microscope, the high concentration of energy at the focal point creates emissions from the cell or cells in that area. These emissions may be read and analyzed to enhance image information and thus improve system control and targeting.

Many MRI and CT systems already incorporate a high accuracy gantry table robot to move the person 3. In a preferred embodiment, the targeting and delivery system are also integrated into the same type of robot to move the person and target area into the field of view and within the range of final aiming. In such an embodiment where MRI or CT technology is used to acquire an image, the robots are incorporated into the MRI and CT system. However, an embodiment of the present invention may use a gantry table robot separate from the MRI and CT system to move the table that holds a person. In certain embodiments, a second level of aiming is accomplished using either mirrors mounted on piezoelectric devices or other technologies such as liquid crystal or plasma deflection. These technologies are able to deliver high accuracy aiming of the energy beam within a small range. System conflict and interference between imaging, control and delivery is reduced or eliminated using gaussian surfaces and other attenuating technologies. High-speed switching between devices may also be considered.

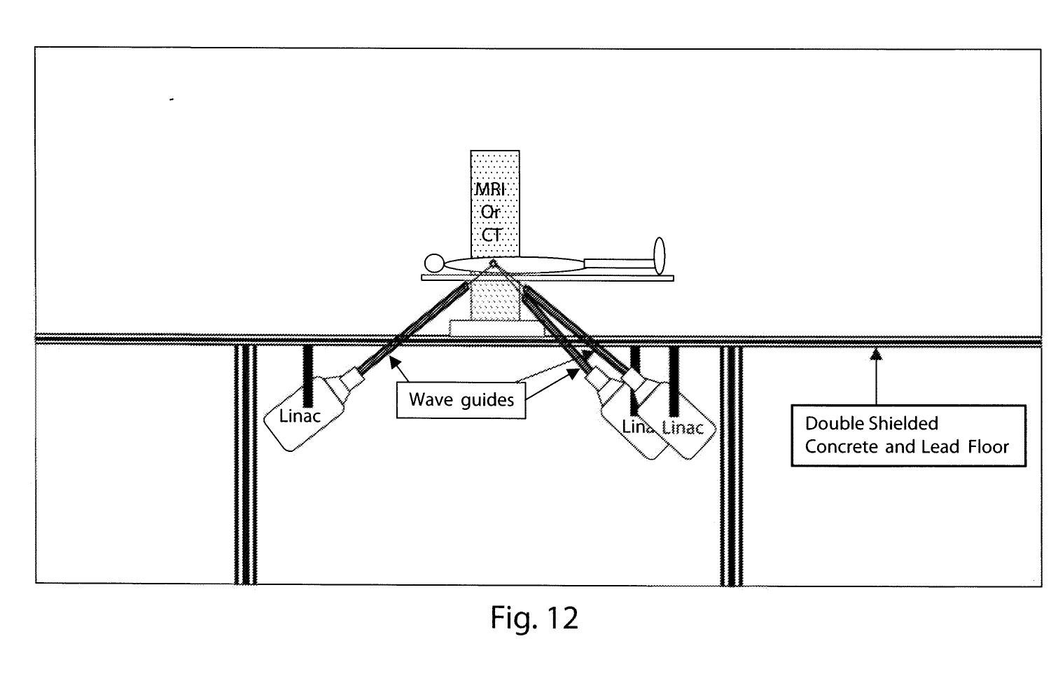

FIG. 12 shows an example of a two story embodiment of the present invention having an MRI or CT image scanner located on a first level and three beam generators disposed on a lower level, capable of transmitting their radiation beams to a person resting on a horizontal platform in the MRI or CT device located on the first level. In such an embodiment a floor design of such a facility housing the design shown in FIG. 12 includes a given of 14 inches of lead or 96 inches of concrete (SR) to block substantially all X-ray scatter. Another given is that there is 24 inches of available space in the floor design. In addition, with an Xray−14 L=0 and Xray−96C=0 where Xray−x*L−y*C=0 and x+y=24 and x is much more costly than y. Based on this information an optimal solution is that y=12.5 and x=11.5. Lead hoods provide a much cheaper way to achieve the desired result. This estimate is based on a worst-case scenario for 100 MeV x-rays. When a preferred embodiment of the present invention is used, lower energy beams are anticipated and therefore require much less shielding, potentially 25% or less of the above estimate. The above analysis is a worst case scenario.

A system block diagram showing the components in a preferred embodiment is shown in FIG. 2. The magnet, RF coils, RF detector and amplifier, MRI pulse generation and magnetic field control and digitizer are MRI components and provide information to a central processor for processing. A preferred embodiment of the present invention includes, among other things, a beam control, digital to analog converter, power amplifier, robot, robotic manipulator and robotic system for controlling the position of the horizontal platform where the person resides during the treatment process, beam generators, aimers, and various device controls. A targeting computer is connected to the central processor. The targeting computer is a sub-processor used to calculate and update feed-forward control instructions and pass them to the central processor. This sub-processor maintains the physical data from the pre-treatment scan and the mathematical model for calculating the feed-forward driver values and gains. This data and model are used by the targeting computer to perform the mathematically intensive calculations necessary for feed-forward control. After completing the calculations the feed-forward values are passed to the central processor and updated feedback information is acquired to make the next round of calculations for feed forward control. The central processor runs the actual control loop with inputs from the targeting computer as well as from the imaging device and other sensors. The preferred embodiments of the present invention use dynamic gains for the best possible control architecture. Any of these gains may go to zero including the gains associated with feed-forward control or feedback control; however, both types of control cannot be zero at the same time. This effectively means that the present invention can run with just feed-forward control or just feedback control. This scenario of mono-tactical control is unlikely to last for more than one second at a time.

A preferred embodiment of the present invention uses aiming technologies similar to those used for industrial robots equipped with image acquisition for the first level of targeting. The second level of aiming would use technology similar to that used in precision machining or high definition television. In each case, feed-forward control strategies would be used that anticipate movements, deflections, diffractions and other error introduction. For instance, breathing is cyclical and movement can be predicted within certain parameters. Bone density compared to other tissue density can be expected to cause deflections and diffractions within certain limits. If the control system model anticipates these in feed-forward control, the feedback loop will be much more accurate.

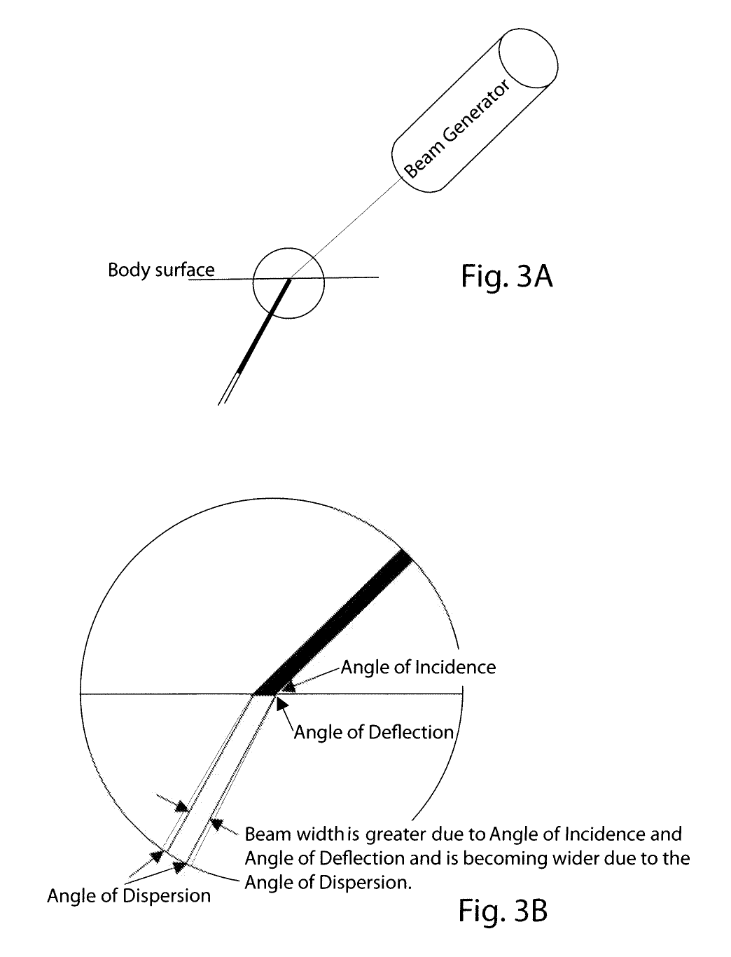

Deflections of x-ray beams have generally been considered negligible in medical applications of the past. The general rule is that the beam will be deflected up to one part in 10,000 per deflection. In other words, at a length of one decimeter from the point of deflection the displacement would be on the order of 10 micrometers. Multiple deflections would be cumulative and may result in much larger displacements. FIGS. 3A and 3B show issues encountered when using a preferred embodiment of the present invention such as the angle of incidence of a beam when it comes into contact with parts of a person's body including bones, tissue, ligament, tendons, organs, and related body parts. These figures show that the effect of the angle of incidence is reduced as the beam size becomes small relative to the size of the cells. The surface of the body can no longer be approximated as smooth or flat on this scale. Rather, the surface of the body is considered to be irregular and covered with things like hair and other obstacles. These surface irregularities will require compensating measures such as shaving and coating. The feedback loop together with the feed-forward model in the control system of an embodiment of the present invention compensates for the residual deflections. More specifically, FIG. 3A shows a beam generator and the beam from the generator coming into contact with the person's skin. Upon doing so a certain deflection occurs. FIG. 3B focuses on this deflection in more detail. The angle of incidence is shown to be the angle when the beam comes into contact with the person's skin surface. The angle of defection is shown to be the angle under the surface of the body (e.g., the person's skin). Most noticeably, the angle of incidence and angle of deflection, when added together, are less than 180 degrees to indicate the deflection the beam made when it came into contact with the surface of the skin. A person of ordinary skill in the art will readily understand that such deflection may have caused the beam to move to the left of the beam when looking at FIG. 3B thereby making such combined angle greater than 180 degrees. In either situation the surface of the body causes the beam to deflect in a certain direction therefore such obstructions need to be accounted for to properly treat the focal point and target area in an effort to minimize damage to any healthy tissue. In addition, FIG. 3B also shows that the width of the beam after it comes into contact with the surface of the body is greater due to the angle of incidence, angle of deflection and angle of dispersion as shown in FIG. 3B as the beam encounters other body parts inside the person's body.

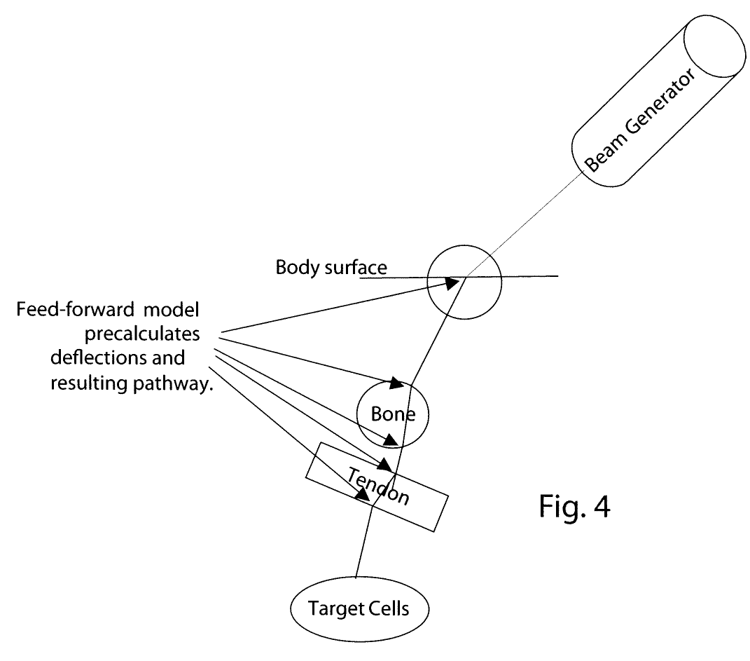

When using a preferred embodiment of the present invention, the displacement tolerance is on the order of 2 micrometers so deflections must be taken into consideration and corrections must be made. Deflection displacement is greater for all other energy types compared to ionizing radiation. As a result, given that displacements for the energy type with the lowest deflection ratio are sufficiently large as to require correction, all energy types will require feed-forward control to compensate for deflections. FIG. 4 shows an example of the feed-forward control system in use and certain deflections related thereto including the person's skin, bones, and tendons before reaching the focal point and target cells. In such a situation, a software program in the targeting computer shown in FIG. 2 includes a feed-forward model that precalculates the anticipated deflections and the resulting pathway and a feedback control for gathering information obtained by the imaging device. Consequently, a resulting system using a preferred embodiment of the present invention is capable of automated, real-time image acquisition, analysis, and treatment. For beam sizes of 7 microns in diameter, the speed at which the system operates is in the range of 10 to 1,000 cells per second.

The preferred embodiments of the present invention provide energy threshold reduction and improvements in accuracy, precision and speed over the most advanced technologies on the market today. In preferred embodiments, the present invention offers the advantage of smaller beam size and lower energy beams, which will also enhance system performance by reducing signal noise for the imaging equipment.

Energy threshold reduction is achieved by avoiding use of hyperthermia to destroy cells. Instead the embodiments of the present invention seek to use the cell's own destructive mechanisms. This results in much less energy being required to destroy the cell and leaves very little cellular residue behind. Attacking the lysosomes, mitochondria or other organelles provides a far more sophisticated approach than simply burning tissue. Tissue ablation or burning is a strategy of last resort for the present invention because of the higher energy requirements and the potential for creating scar tissue inside the body.

Accuracy, precision and speed are improved directly and dramatically by the use of feed-forward control combined with feedback control. Feed-forward control also provides work area size reduction so that the amount of data processed in each repetition of the feedback control loop is minimized. This minimization creates a much faster feedback loop, which again improves accuracy, precision and speed of the total process.

The initial precision and accuracy of the preferred embodiments of the present invention are approximately 7 micrometers±2 micrometers, which is slightly smaller than the size of the smallest human cells. This is approximately 50 times better in each axis than any competing technology. The technology used in the present invention has the potential to improve the precision and accuracy by another order of magnitude as imaging and beam generation technologies improve. The technology described herein related to preferred embodiments of the present invention provide a potential cure for many currently incurable diseases. It also provides a quantum leap forward for many areas in which cures or treatments already exist.

Conventional treatments for cancer today are very crude in comparison to the treatment from the preferred embodiments of the present invention. Current treatments use a single relatively broad beam of radiation that causes damage to surrounding tissue. There is a delay between acquiring the image of the problem area, diagnosis, and any action that may be taken. This delay can be significant and mean the difference between life and death for a person. Repeatability is low and the opportunity for human error is high. Targeting and aiming are limited to the most rudimentary methods. Targeting of certain cells or very small groups of cells is non-existent (compared to the present invention).

Other areas of healthcare in which the device may be used would see the benefits of greater accuracy and repeatability. Elimination of human error through the use of automation and real-time technology would be significant. The technology described herein also offers the substantial benefit of being non-invasive therefore avoiding surgical procedures. Other uses include enlarging respiratory passageways, repairing heart valves, reducing prostate size, improving hearing, stimulating brain cells, internal cauterizing to stop bleeding, treating fatty liver syndrome, and removing polyps.

The preferred embodiments of the present invention work autonomously after set up and include at least the following benefits: error reduction, improved repeatability, greater accuracy, faster operating speeds and better tracking. Automation makes the system work in a beneficial manner because the selection and targeting of cells is computationally intensive. Aiming beams must be very fast and highly accurate. For a human to make an informed and accurate decision for one cell may take hours. It would be tedious and errors would be unavoidable. Even if the problem of human error may be overcome, persons would not be able to endure the length of non-automated procedures. Even small movements in the body make it very difficult to keep track of the area being analyzed for periods lasting more than a few milliseconds. Analysis and action is done together in real time.

The image information is also more meaningful numerically than it is visually. In the case of the MRI system the mathematical space (k-space) in which the image is acquired is transformed and interpreted into pixel information for the human eye to see. These mathematical operations can introduce tolerance errors. The visual representation of the information then is also subject to limitations of the eyes and mind of the person looking at it. Automation is objective, repeatable, fast, accurate, and reliable. For the work that must be done once a procedure is started these characteristics are more than highly desirable they are a requirement. As discussed above, FIG. 2 provides a flow chart of a preferred embodiment of the present invention and includes such components to ensure the system is automated and therefore more effective in performing the treatment for the person.

Further to the description above regarding beam generations, although radiation beams are the logical choice for a preferred embodiment of the present invention, radiation beams are not the only choice and depending on the particular application, are not necessarily the best choice. The architecture in the present invention makes it possible to work with any energy beam that penetrates flesh. Radio waves, ultrasound and other energy beams may be used with the preferred embodiments of the present invention. Various wave lengths/energy levels of electromagnetic beams (photons) and mechanical waves (ultrasound) can be used with the present invention. Ionizing radiation has some significant side effects and risks. Even with the use of low power beams, radiation may not always be the best choice for every application. Other energy beams may be more effective in terms of focus, penetration, energy delivery, safety, destructive capability and/or side effects.

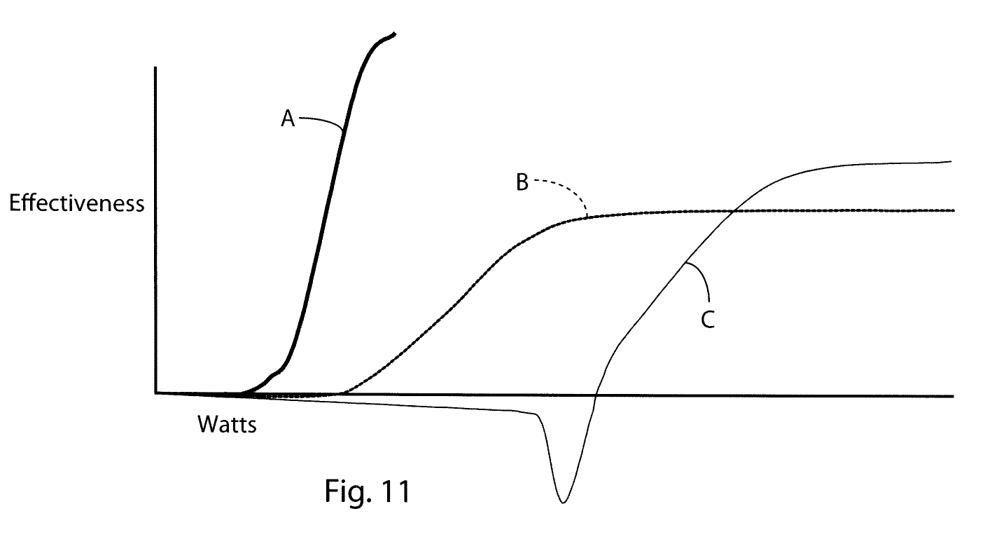

A preferred embodiment of the present invention uses a combination of radiation beams based on the criteria of obtaining maximum effectiveness with minimal side effects. A preferred embodiment needs only to rupture the cell membrane or disable organelles to kill the cell. A preferred embodiment as discussed above causes the cell to self-destruct and dissolve itself from within. Rupturing the cell membrane may leave behind cellular material for decay and potential infection. Tissue ablation can leave behind scar tissue. Dissolved material will be more easily absorbed and reused or discharged from the body. Radiation has the effect of degrading or decaying the cell membrane until it ruptures. Focused, intersecting energy beams controlled by the present invention are used, among other things, to heat the organelles (such as the lysosome or mitochondria) or cellular fluid to cause disability of the organelle and cell death. In the same way, the preferred embodiments of the present invention make it possible to use ultrasound to vibrate the lysosome or other cellular material at an energy level sufficient to cause cell death. The trade off is the side effects (or amount of the damage) the individual beam type has on the various entry and exit pathways. To get sufficient penetration, the microwave beam strength would have to be too strong to avoid damage at the entry surface in order to reach cells deep within the body (more than 3 or 4 centimeters). FIG. 11 shows three effectiveness curves, i.e., curve “A” shows an effectiveness curve created when the present invention uses three beams and nanoparticles are attached to the organelle within cells; when using this preferred embodiment 100% cell death occurs in the target area. Curve B shows an effectiveness curve created when the present invention uses a single beam and nanoparticles are attached to the organelle within cells; when using this embodiment approximately 50% cell death occurs due to the limitation imposed by using only a single beam. Curve C shows an effectiveness curve using conventional radiation, which shows that under certain circumstances such a conventional radiation treatment may actually result in negative effectiveness in the human body known to cause cancer. Curve C also shows that a certain plateau is reached due to the death of cells related to healthy tissue around the target area/focal point. It is noted that these curves show expected results based on calculations preformed by the inventor, i.e., these curves are not based on experimental data.

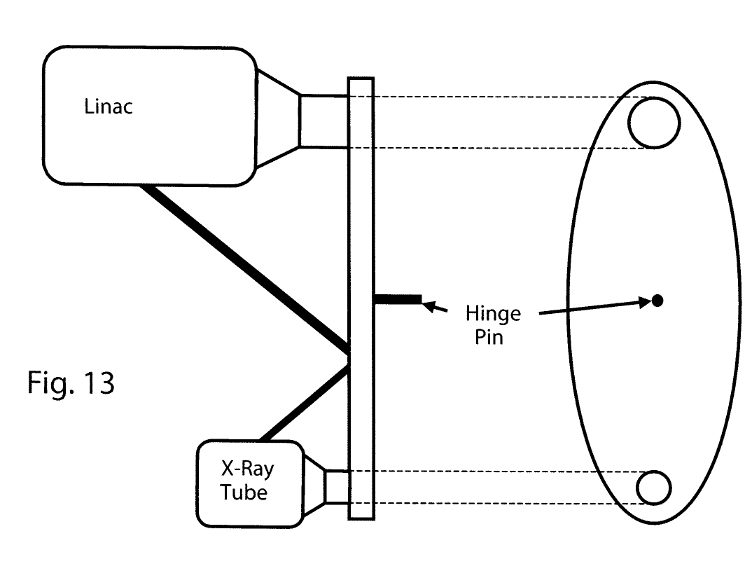

The beam generator for a preferred embodiment of the present invention is capable of generating several different types and sizes of beams based on the preferred application. The various beam types may be used as alternatives or in combination to achieve optimal results. Multiple energy types combined in one beam will provide the best results in terms of the amount of energy required to achieve the desired results. FIG. 13, for example, shows an x-ray beam used in combination with a beam generation unit.

Harmonics imposed on or modulated in the primary waves may also be used to reduce the energy required to achieve the desired results. Harmonics, matching the size of molecules within the organelles, entire organelles, or the entire cell, will cause faster energy absorption and therefore result in less energy being required. Because wavelengths of flesh penetrating beams are very short (shorter than the harmonic wavelengths needed), the harmonics may be achieved by modulating the release of energy in the beams. The use of harmonics will make targeting of organelles much easier in that the beam intersection point will only need to include the organelle rather than be focused on it.

Beam size and energy levels as they relate to imaging. In general, a person of ordinary skill in the art will readily understand that imaging equipment such as an MRI or CT is very sensitive to stray energy. Compton and Thompson scattering of x-rays make the concurrent use of X-rays and imaging equipment challenging. Compton scattering is the primary cause of image distortion because it is the primary cause of scattering and results in photons being directed in random directions. Scattering is caused by a photon colliding with an electron and being absorbed by the electron temporarily. This causes the electron to exit the atom or jump to a higher shell leaving an empty position in its original shell. When an electron drops back to the vacancy in the original shell it emits a photon in a random direction. Some of these photons will interact with the sensor array. Reducing the beam energy and size reduces the scatter. The fewer the input photons per unit of time the less the scatter per unit of time. Given that the image acquisition takes a fixed amount of time, the reduced scatter per unit of time means less image distortion.

Reducing the energy level of the photons being shot into the person also reduces the scattering. Below a threshold of 14.32 KeV the X-ray photons can only expel electrons from the L and M shells. Photons emitted as a result of an electron dropping back to the L or M shell are much less energetic and less able to penetrate flesh. These interactions are also much less likely and therefore less frequent. As a result, few of these photons will reach the sensor array. The Applicant is in no way limiting the embodiments of the present invention to those energy levels below 14.32 KeV. Instead, the Applicant suggests that there is a benefit to lowering the energy level in the beams to reduce scatter and the resulting image distortion. Scatter and distortion are related to the energy level of incoming photons. This relationship is not linear but the existence of the relationship means that there is one or more optimal energy level(s) for photons used in the present invention. Other criteria for determining the optimal energy level are the ability to penetrate flesh and the risk to patients and healthcare workers.

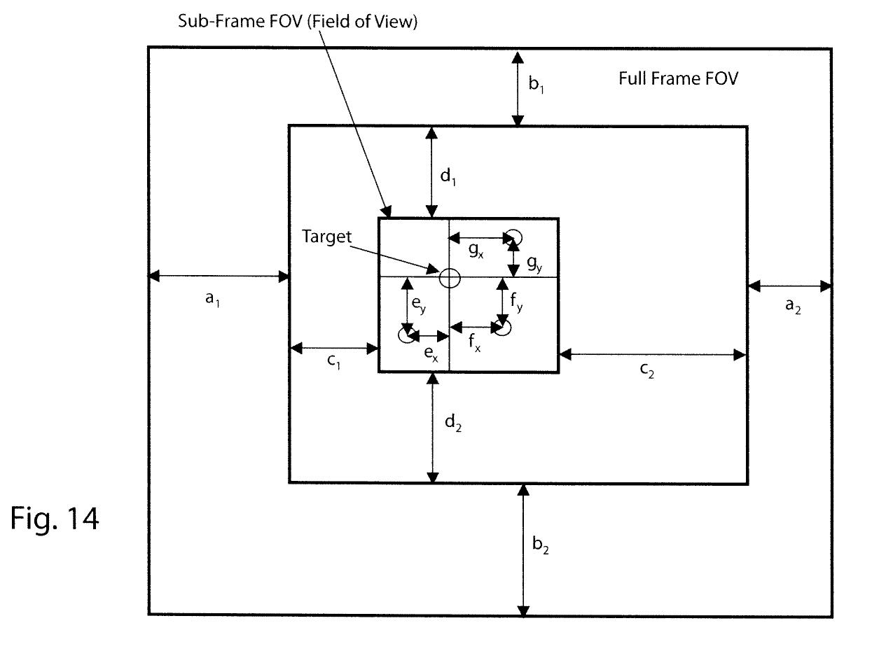

FIGS. 14-19 show features of the preferred embodiments of the present invention related to the targeting and aiming of the energy beams. As understood by a person of ordinary skill in the art, “targeting” is the selection of target cells and “aiming” is the guidance for delivering energy to those targets. Targeting requires a pre-scan plus real-time scanning. The pre-scan provides information to the automatic modeling process needed for the feed-forward control as well as the input for the graphical user interface (“GUI”) where doctors select targets and provide setup parameters. The setup parameters define the space limits within which the system can operate. Potential targets are identified by the targeting computer in the pre-scan and are presented in the GUI so that a doctor can select which potential targets become final targets.

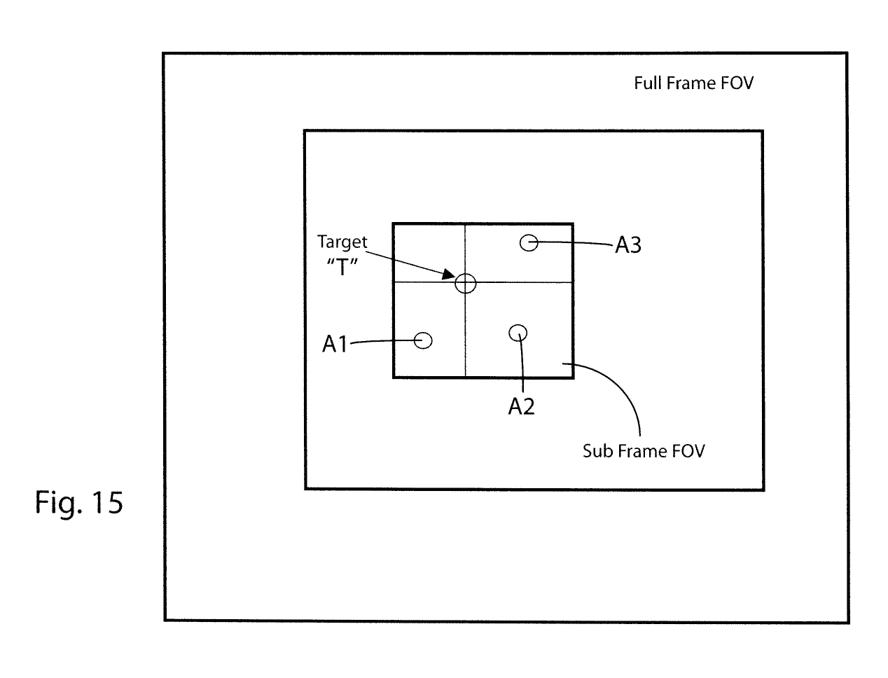

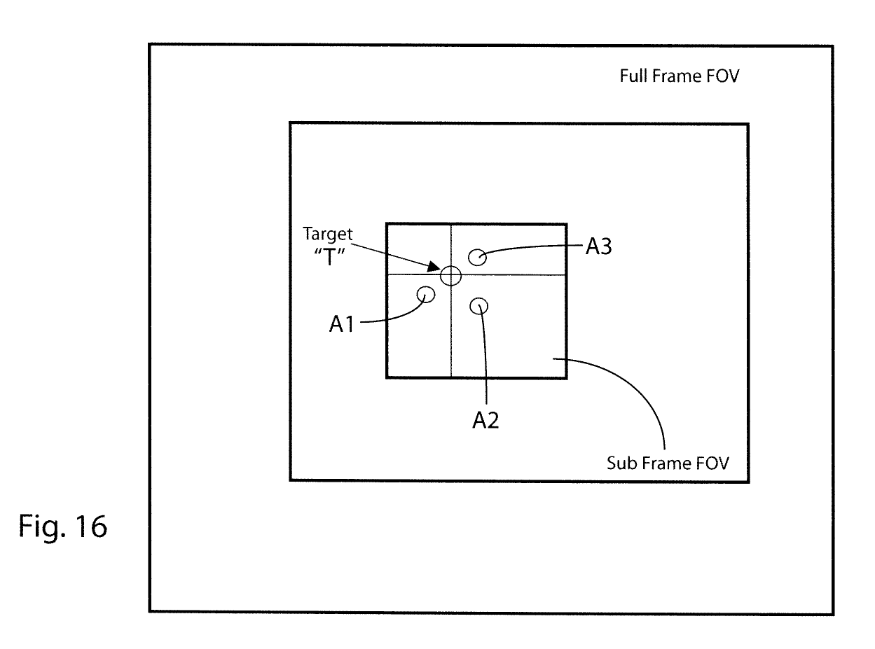

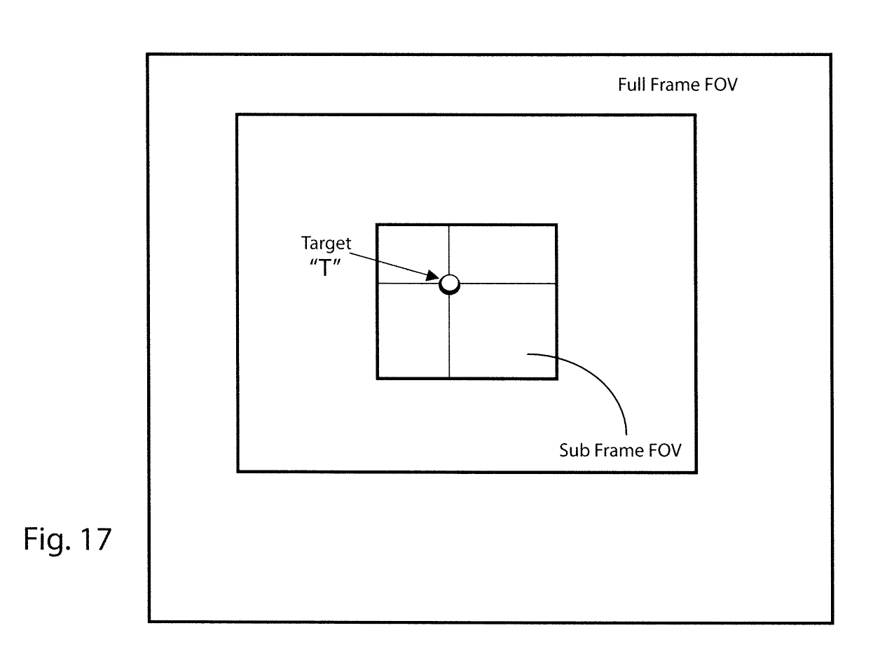

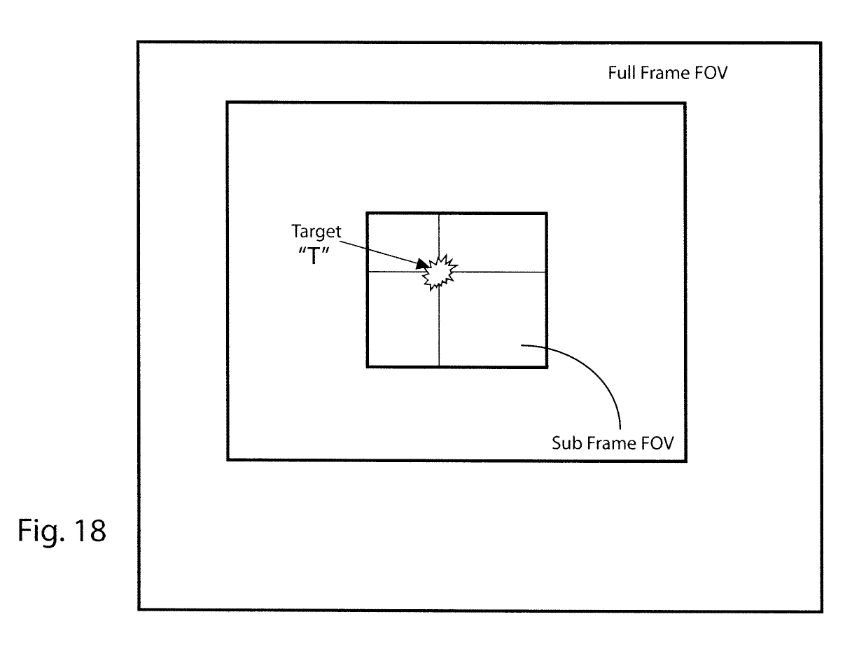

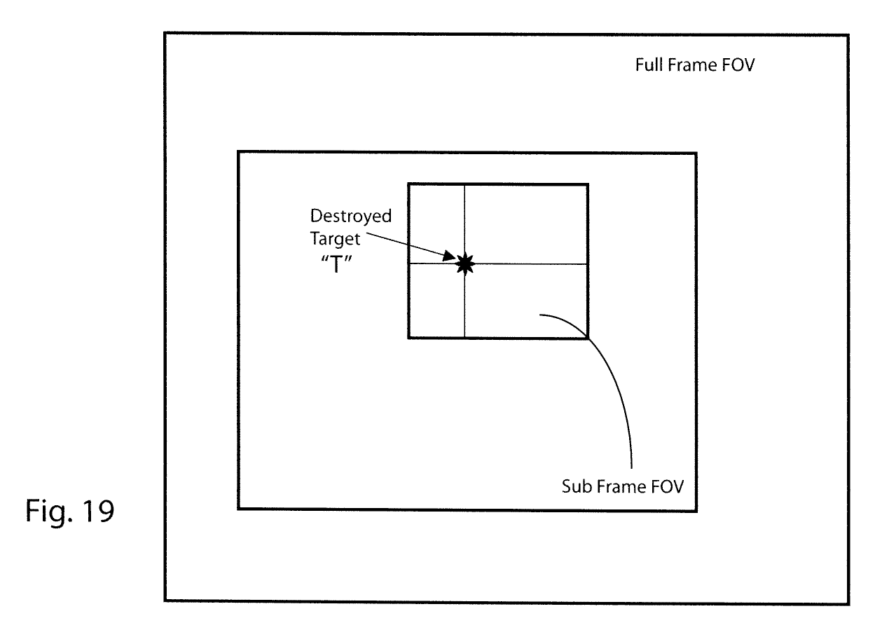

The feedback control loop used in aiming the beams preferably uses visual data from the image acquisition system. It may be necessary to modify standard imaging systems such as an MRI to enhance the visibility of the beams in the image data. Tracer elements (such as additional wave lengths) may also be included in the beam generation to enhance visibility of the beams. Compton and/or photoelectric scattering make high-energy x-rays visible to CT or PET equipment. FIGS. 14-19 show a sequence of six frames of a control sequence for a preferred embodiment of the present invention that takes into consideration errors in the initial aiming of the beam generator in relation to the target, uses the feedback error values in conjunction with the feed-forward control to adjust the beam until the beam converges on the target area and after releasing the beam pulse, to destroy the target cell. More specifically, FIG. 14 shows a full frame field of view and a sub-frame field of view for the target area on the imaging device associated with the present invention. The reference letters “a₁,” “a₂,” “b₁,” “b₂,” “c₁,” “c₂,” “d₁,” “d₂,” “eₓ,” “e_{y},” “fₓ,” “f_{y},” “gₓ,” and “g_{y}” represent feedback errors caused by the three beams A₁, A₂ and A₃ not converging on the target “T.” For example, the references “a” and “b” may represent robotic arm errors, references “c” and “d” may represent gantry table errors and references “e,” “f,” and “g” may represent final aiming errors. These reference error numbers are used in conjunction with the feed-forward control to ensure the beam converges on the target area so that when the beam pulse is released on the target “T” the target cell is destroyed, not the healthy tissue surrounding the target area. FIG. 15 shows the specific target “T” and location of each of the three beams A₁, A₂ and A₃ around the target “T.” As shown therein, FIG. 15 shows that the three beams A₁, A₂ and A₃ have not converged on the target area T. The spacing between the three beams A₁, A₂ and A₃ and target area T are calculated, and are used to manipulate a deflection device at each beam generator. FIG. 15 shows another example of the target area “T” in relation to the three beams A₁, A₂ and A₃ FIG. 15 shows that the spacing between the three beams A₁, A₂ and A₃ and target area T are getting smaller as a result of the deflection devices causing the beams A₁, A₂ and A₃ to move closer to the target “T.” FIG. 16 shows an example of the three beams A₁, A₂ and A₃ converging on the target “T” until any error is within a certain acceptable tolerance level and FIG. 17 shows the three beams A₁, A₂ and A₃ converged on the target “T.” FIG. 18 shows an example of the beam pulse being released after the beams A₁, A₂ and A₃ have converged on the target “T” therefore destroying the cell. FIG. 19 shows an example of the system verifying that the target has been destroyed.

Target vs. Non-Target Differentiation: In making determinations between target and non-target differentiation, mathematical or control differentiation between target and non-target cells is one of the critical issues especially when target cells are physically close to sensitive, non-target cells such as nerve cells. Making the target cells look and/or react substantially different is a challenge. To achieve this differentiation there are several approaches that may be used: 1) nanoparticle adhesion to target cells that reduce the energy needed for cell death, defining margins around sensitive areas within which no targets may be selected, as described further below; 2) use of marker dyes on target material/cells; and 3) use of mathematical algorithms in the control law that enhance differentiation between target and non-target material, as described further below. The very small beam size and the control architecture in the preferred embodiments of the present invention are strategies for assuring that target cells and only target cells are destroyed.

Heat Dissipation: Heat dissipation inside the body may be a problem especially for applications that require a large amount of work in a concentrated area. To avoid unnecessary heat build-up the preferred embodiments of the system will self optimize to deliver the minimum amount of energy required to cause the desired effect. This feature is part of the automatic modeling used for feed-forward control. The amount of energy required to rupture the mitochondria, lysosome or other organelle is likely to be sufficiently small such that the heat from the operation will be easily dissipated naturally by the body's own systems. Some procedures may require the use of auxiliary cooling such as an endothermic or cooled IV. Scattered targeting to avoid too much energy being released within a given space may also be used. The simplest solution would be to slow the system down to meet the body's ability to dissipate the heat naturally. This will work for some applications but may cause the overall length of other applications to be intolerable.

Using the mitochondria, lysosome or other organelles to dissolve the cell: Harnessing the power of the lysosomes to dissolve the cell will result in less energy being used to accomplish cell death. The complexity is that simply rupturing the lysosome will not work because the enzymes within the lysosome require a low pH level to be activated. The normal pH level within a cell is too high. One strategy to engage the lysosomes is to attack the mitochondria. If sufficient damage is done to the mitochondria it will trigger digestion of the cell. In essence, destroying the mitochondria kills the cells and causes the breakdown of the cell into elemental components. Destroying all or almost all of any type of organelle within a cell will accomplish cell death.

A preferred method for targeting the mitochondria is to use gold or carbon nanoparticles with targeting agents attached. One method includes attaching peptides to the nanoparticles that will seek out and attach to the mitochondria of certain cells. Another method includes use of monoclonal antibodies attached to the nanoparticles to bring the particle to a specific cell type (target cell) plus use of an attached peptide chain to cause the particle to lodge in a pore of the mitochondria of the cell so that beams of energy can be used to activate the particle such that the mitochondria is ruptured and apotosis is initiated to destroy the cell. An alternate to adding a monoclonal antibody to the nanoparticle is to add an aptamer. An aptamer is an oligonucleuotide of DNA, RNA, or a modified DNA or RNA. It is short (10-15 nucleotides in length) and binds specifically to certain proteins. Approximately 200 have been characterized to date. One has been discovered that binds to liver cancer specifically. The aptamer that was characterized for hepatoma is recognizing and binding PDGF alpha which is normally only expressed in embryos. A preferred embodiment of the present invention may use this aptamer attached to a nanoparticle to target liver cancer. Another aptamer which has been described recognizes prostrate specific membrane antigen. Another possibility is to use Macugen which is an aptamer developed by Eyetech, Inc against VEGF. VEGF is overexpressed in tumors because of the requirement for neovasculariztion.

Nanoparticles are targeted to diseased cells via attached peptides, antibodies, antibody fragments or aptamers. The nanoparticles also have a mitochondrial targeting peptide attached to send the nanoparticles, once in the target cell, to the mitochondrial pores of the mitochondria. The nanoparticles will plug the pores, as their size will be slightly larger than the pore size, such that they fit snuggly in the pore. The photons will then energize the nanoparticle such that it creates a hole in the mitochondrial membrane, allowing release of cytochrome “c.” Cytochrome “c” release into the cytoplasm will trigger apoptosis, or cell suicide, initiating degradation of the cell from within. The present invention uses nanoparticles that include gold, carbon, iron, magnetic material, compound metal, tubes, balls, bubbles, springs, coils, rods or combinations there of.

Effect of Beam Size and Wavelength on Deflection Considerations: High energy x-ray beams are usually modeled as being unaffected by passing from material of one density to material of another density. This model works well for large diameter beams, as the actual deflections are small compared to the beam size. However, as the beams size and target size become smaller (as in the present invention) the small deflections become more significant. Even a very small angle of deflection will move the beam. Because the target is very small and the desired intersection point is equally small, these small deflections cannot be ignored.

The shorter the wavelength is the less the deflection is. This effect is readily seen in rainbows and is where deflection meets diffraction. As the wavelength approaches zero the deflection will also approach zero. This causes different wavelengths to separate and travel in different directions. In the case of polarized light, we see the colors of the rainbow. In the case of non-polarized, non-coherent x-rays, we see seemingly random dispersion (at small angles). In the case of ultrasound, we see dispersion, phase shift and even changes in wavelength. To achieve predictive results the energy beam must be refined. For x-rays the use of a waveguide provides a known in the art method to produce a coherent beam. As little as three feet of lead with a straight passageway corresponding to the beam size will deliver the desired results. Adding a filter to absorb the low energy photons and choosing a source that emits x-rays not to exceed an upper limit of energy (say an x-ray tube) the beam can be made highly homogenous. An alternate and more precise method would be to deflect the x-ray beam (say from a Linac) in such a way as to select only a certain wavelength to enter the waveguide.

Beam trajectory selection/beam generator articulation/patient articulation: If we assume that we use a preferred embodiment of the present invention includes three orthogonal beams with an intersection point falling within the active section of the imaging system, the beam generators do not need articulation beyond what is provided by the final aiming device. The gantry table system used in the MRI device to manipulate the position of the person will provide the full six degrees of freedom needed to position the person for treatment. However, there are parts of the body for which three orthogonal beams will not provide optimal pathways to the target. In a preferred embodiment, to optimize each pathway individually a minimum of two of the beam generators would need to have the capability of six degrees of freedom of movement. The determination for choosing the optimal pathways are based on protecting sensitive tissue, avoiding complicated obstacles, and minimizing total beam energy needed to achieve the desired results.

Beam trajectory Deflections and other Calculations: As part of the preferred embodiments of the present invention, the feed-forward control, beam pathways, deflections, absorptions, attenuations, diffusions and resulting robotic controls are pre-calculated. The pre-calculated movements, torques and motor currents required for the various robotic system components are functions of the pathway, deflections, absorptions, attenuations and diffusions. These pre-calculated movements combined with feedback control yield precise and accurate placement of the intersection point within or encompassing the target.

Singularities: Singularities are control issues that are mathematically indeterminate. They are usually caused by division by zero in automated calculations or by calculations that result in multiple solutions. The preferred embodiments of the present invention are inherently prone to singularities. To resolve singularities several strategies will be considered. One of these is to include sequenced priorities for movements and trajectories. In other words, the first time a target is approached from a standard trajectory will be different from the second, third, fourth . . . . These standard trajectories would also include standard movements and thus eliminate most singularities.

As discussed above in relation to FIGS. 3 and 4, the calculations for the pathway selection are a function of obstacles, distances and densities within the tissue surrounding the target. Obstacles include sensitive tissue that should be avoided. These obstacles' distances and densities are well known within certain parameters for normal human bodies (not deformed or injured) and can be quickly verified in the pre-scan of the patient. This knowledge base will be used to reduce computing time in the planning and targeting processes. At each transition point from tissue of one density to another density a deflection angle is calculated working backward and forward from the target. There may be multiple deflections creating a complex path for the beam. To simplify this process and reduce or eliminate singularities, standard pathways for each type of procedure will be used for targets within each region of the body. The total number of regions needed in the body for the purpose of pathway selection is currently unknown but is likely to be more than one hundred. The standard pathways will be defined with tolerances for automated adaptation for patient specific applications.

Bone and Tissue density calculations: Bone and tissue density are calculated in the pre-scan and are used in targeting and trajectory planning. The imaging system may need to be adapted to acquire density information. Additional sensors with inputs to the system may be required. Age, gender and health issues will be inputs to the system to be used to expedite the process and reduce the computational load for determining densities. CT PET scans may also offer valuable information about density as the molecular make up of bones (and to lesser extent flesh) is an indication of density.

Entry simplification (submersion, gels): For very narrow beams of ultra sound (and potentially other types of energy) it may be necessary to simplify the entry surface of the body. Irregularities in the surface of the skin may cause unpredictable and large deflections. Most of this type of error introduction may be reduced or eliminated by use of coatings or by submerging the body in water. The coatings or water would have the same density as the skin so there would not be any deflection (or minimal deflection) when crossing from one into the other regardless of the surface irregularities between the two materials.

Energy type speeds: Ultrasound, microwaves and radiation travel at known but differing speeds through materials of a given density. For complex beams composed of multiple energy types, staged firing would be required to cause the various energy types to arrive at the target at the desired time and sequence. It may be more desirable for one type of energy to arrive slightly before or slightly after another type of energy or it may be best to have all of the energy arrive simultaneously to achieve threshold energy levels. In a preferred embodiment that includes the sequenced arrival of the energy, the desired effect would be to lower the individual thresholds for each subsequent energy type. For example, radiation may be used to weaken the cell membrane followed by microwave to heat and expand the cell followed by ultrasound to vibrate the weakened cell to a quick collapse. In the case of simultaneous arrival, the desired effect would be to quickly cross the threshold for total energy required to create the desired effect.

Creating an Intersection Using Speed Differentials: Even a single beam type travels at varying speeds in different substrates. This becomes even more complex as different beam types travel at differing speeds within a single substrate. For instance, ultrasound travels much slower than radiation within any given material. In addition, ultrasound travels at differing speeds as it passes from one material to another.

The preferred embodiments of the present invention account for these variations in speed within its feed-forward modeling. However, the present invention may also make use of these variations to create intersection points for two or more energy types emitted from a single beam generator. This is accomplished by releasing the slower moving energy type/beam first and then releasing the faster beam such that the faster beam catches the slower beam at the target and creates a high energy intersection point as the bursts of energy converge.

Using tumor density for target selection and aiming: Tumors and cancer cells in general have different density characteristics than normal healthy cells. These characteristics can be used to help in the selection and destruction of targets. Energy beam absorption and image contrast are helpful characteristics for use with preferred embodiments of the present invention. Use of an energy level feed back loop with the present invention may provide significant improvements in aiming the beams relative to the tissue density and thus result in improved accuracy and/or speed.

Tracking of energy beams through the image acquisition system may be enhanced if the beams contain ions, as the charge and resulting electromagnetic fields should be visible to the detectors used for magnetic resonance. On the other hand, the charged particles will also generate their own magnetic field as they pass by the detectors which will create some image distortion or interference. It may be possible to compensate for the distortions mathematically or through other means such as image subtraction or exclusion.

As two beams containing ions become close to one another, trajectory deflections will be a complexity for the control system. Particles with like charges will repel one another while opposite charges will be attracted. These forces will have some impact on beam trajectory if the particles remain in the beam after entering the body.

Digitization scheme (priorities for visual analysis compared to computational analysis)—Modifications of the MRI system architecture: Direct digitization rather than frame grabbing will be necessary to achieve the best possible image resolution and avoid video jitter. Conversion of source information into a standard video signal (RS170) and then using a frame grabber and digitizer introduces error and causes information to be lost. Reconfiguration/modification of the imaging hardware to provide direct digitization will be required to improve system performance for the present invention.

Wave Cancellation and Amplification: If the energy beams are modeled as continuous, homogenous waves, phase shift control in the energy waves seems critical at first glance. Phase shift control is needed to assure that maximum energy is released at the point of intersection. Wave cancellation or amplification occurs when waves intersect at inverse points in their curves. To achieve optimal energy delivery to the target point, a person of ordinary skill in the art will appreciate that phase shift needs to be tightly controlled. However, phase shift is only relevant for coaxial beams or beams for which the axes are offset by a small angle. As the angle between the beams becomes larger the effect becomes smaller until it reaches zero effect at 90 degrees. At 90 degrees there are areas of total summation and other areas of total cancellation in every repeatable element of the intersection regardless of phase shift.

If the beams are modeled more appropriately as sub-atomic particles (photons) colliding with electrons, then a different conclusion is reached. The rational for summation at the intersection point becomes clearer. In fact, the known ways in which x-rays interact with atoms suggest very little or no cancellation in the intersection point. Compton effect and thompson effect should both release energy in the intersection point. Add to these the potential collision of high-energy photons and resulting release of energy.

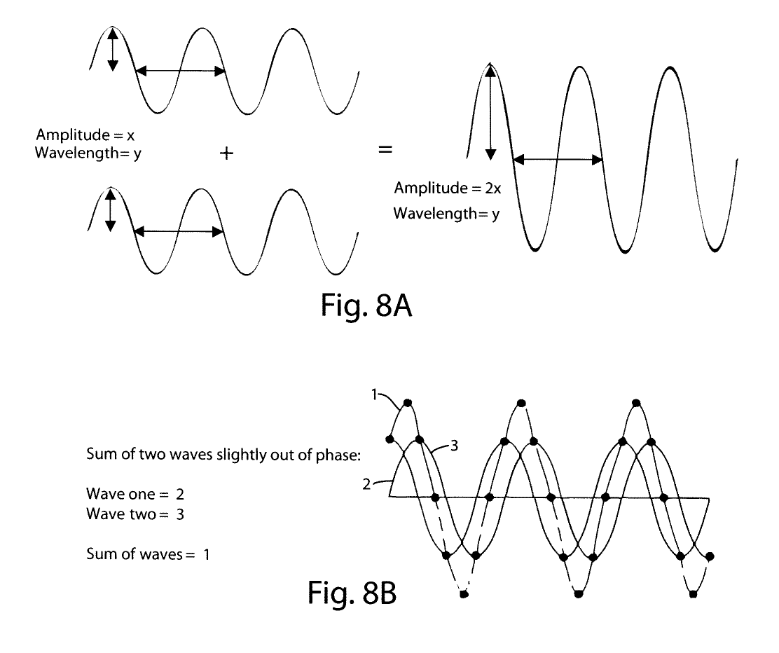

FIGS. 8A, 8B and 10 provide information regarding the energy waves sent from the beam generators to destroy the target cells. An understanding of the characteristics of the energy waves and their amplification inside the intersection point will provide a person of ordinary skill in the art a better understanding of the waves' effect on the target cells. For example, FIGS. 8A and 8B include information related to the wave amplification inside the intersection point. FIG. 8A shows on the left that when the two waves are separate each wave has an amplitude “x” and a certain wavelength “y”; when those two waves are in phase and added together they have an amplitude of “2x” and a wavelength of “y.” FIG. 8B shows that if the two waves are out of phase and added, waves 1, 2 and 3 are created at the intersection point. FIG. 10 shows an electromagnetic wave that includes a magnetic field, an electric field, a certain wavelength of the electromagnetic wave and the propagation direction of the electromagnetic wave. The wave in FIG. 10 shows that at the beginning of each wave a step up in intensity will occur and at the end of each wave, a step down in intensity will occur. Most importantly, FIG. 10 demonstrates that the magnetic field and electric field will both be amplified in the intersection point.

Focal Point Concentrations: Focal point concentrations would occur on the surface of the body closest to the beam generators if the person or beam generators were not moved between shots of energy. This effect is seen if the target requires multiple shots and only one or two angles are adjusted between shots by final aiming. This creates a cone defined by the circle encompassing the outer most points of the target and the point at the end of the beam generator (vortex). The cross section of the cone (various trajectories) becomes smaller and smaller as the section is moved closer to the beam generator and the maximum concentration is at the surface of the body. As shown in FIG. 4, small movements of the patient/table may cause sufficient variation in the trajectories to avoid problems related to focal point concentrations.

Energy Threshold Considerations: FIG. 5, as mentioned above, shows the general expected results of using multiple beams of ionizing radiation at the focal point. For example, the watts, gradient, cell absorption and cell death are greatest when four beams are used. Like an electron microscope, the high concentration of energy at the focal point will create emissions from the cell or cells in that area. These emissions may be read and analyzed to enhance image information and thus improve system control and targeting.

If z is the minimum energy absorption required to damage a cell, and y is the beam strength/absorption at the point of entry into the body, and x is the beam strength/absorption at the target, and w is the number of beams used, then:

1/w·*z<x<y<z.

The energy gradient must also be considered in determining the rate of absorption. Higher levels of energy are to be expected to cause faster absorption, therefore the energy absorption within the intersection point will be higher than the energy absorption outside the intersection point and will be a function of the number of beams. If m is the energy absorption rate outside of the intersection point, and n is the energy absorption rate inside the intersection point, and w is the number of beams, then:

w·m<n.

Energy Threshold Reduction: the present invention seeks to reduce the energy threshold by using cellular features to assist in the destruction of cells. The exact amount of the reduction in the amount of energy required to destroy a cell resulting from this strategy is expected to be on the order of a factor of 100 as the mitochondria or lysosomes account for less than 5% of the total cell by volume. The work of breaking down the cell is accomplished by the enzymes in the cell rather than energy from the beams.

Control Architecture (FIGS. 14-19): Feed-Forward Control. The preferred embodiments of the feed-forward control for the present invention will pre-calculate the physical characteristics of the process: target locations, landmarks for target acquisition, target size, optimal beam sizes at target, beam path working backward from the target, diffraction angles, deflection angles, beam diffusions, beam strengths needed at target, absorption and attenuation rates along the beam paths, power loss along each trajectory, initial beam strengths required, beam size required at generator, number of beams needed, gantry robot position, robotic positioning of arms for each beam generator, and anticipated movements in six degrees of freedom, person's movement range, person's movement cycle, phase shifts and firing sequences. All of these parameters are used to choreograph the details of the feed-forward control.

The robotic system will recalibrate itself for each still position based on static or quasi-static landmarks within the person's body. If the target or beams move out of the field of view the system will automatically recalibrate and start back from where it lost the feedback inputs.

Feedback Control and Final Aiming. The feedback loop will use digital information gathered from a sub-frame of the imaging equipment. This sub-frame will provide information about a small area around the target and will only be large enough to assure that the target and feed-forward aimed beams are included within the frame. The feedback loop will then control the final aiming of the beams to cause them to converge at the desired intersection points within the target.

For this multi-stage aiming to work effectively the robotic systems will be required to hold a still position relative to the target within a tolerance on the order of one to two hundred micrometers. A preferred embodiment would be for the robotic system to meet these tolerance criteria on its own by use of image feedback. However, this tolerance may also be sequential within the cycle of movement. In other words, there may only be one or two points within the cycle of movement that the still system is within tolerance for the present invention to emit a burst. In this case the image and control phase would need to be shifted to accommodate the movement cycle.

An objective of the robotic aiming is to have the target and all of the beams within 400 to 500 micrometers of the center of the sub-frame field of view. If the field of view of the sub-frame is 4 mm square, final aiming control will have adequate space within which to measure error for each of the beams relative to the target. This error is then used as the input in the control law for the feedback loop.

The control law for the feedback loop converts the error measurements into a usable signal for the final aiming device. In the case of electromagnetic fields being used to deflect the individual beams the control law will generate a series of electric currents with differential values. These currents power the final aiming actuators so as to cause the desired deflections.

The control law gain for final aiming will be variable and automatically adjusted to account for proportional movements. In other words, the movement of each beam must be expected to be proportional to the skew of the magnetic field but the proportion will not be constant. It is expected that beams will move more or less within the field of view for the same deflection at the final aiming device depending on the mediums through which the beam must pass. For example, as shown in FIG. 4, a beam passing very close by a tendon or bone will suddenly have a different deflection pattern if final aiming moves the beam such that it makes contact or passes through the different medium. However, the position of the beam in the field of view will be a continuous function of the final aiming control. So, while the proportion of the movement of the beam is not constant it is measurable and therefore can be adapted in the control law to achieve the desired accuracy (provided the hardware can produce the final aiming movements in increments sufficiently sensitive to cause the desired deflection.) Phase shift adjustments may also be required as a part of the feedback control.

Image Control: Sub-frame images will be used to achieve speeds and accuracies desired for the preferred embodiments of the present invention. This is achieved by processing a small portion of the array at the maximum accuracy. While the total array for the slice may be forty to sixty centimeters squared, the sub-frame to be processed for feed back control would be on the order of three or four millimeters squared. The elemental information collected about each pixel by various sensors is distributed over a large portion of the sensor array. So, to gather useful, complete information about a sub-frame will still require partial processing of a portion of the sensor array that is larger than what may be expected on an intuitive level.

Robotic Stability Requirement: For the system to work the robotic systems will be required to produce a stable coordinate system relative to one another with a tolerance on the order of 0.1 micrometers per feedback loop cycle. This design criterion is dependent upon the speed of the feedback control loop. The faster the loop, the larger the tolerance. The limiting factor for the feedback control loop speed is the MRI frame rate. Published frame rates for MRI systems are on the order of 10 frames per second for high accuracy images. Gyroscopes on the robotic end-effectors (beam generators) may help achieve this design criterion.

To improve the speed of a procedure the system is equipped with the capacity to vary the diameter of the beams. Larger beams may be used to more quickly eradicate larger groups of cells. Smaller beams will be used to target smaller groups of cells or individual cells.

The system will take a significant amount of input from a doctor to set up for each patient and will operate only within parameters the doctor sets. However, the process will be highly automated once it is started. Failsafe measures, including an emergency shutdown button, will be included in the device.

We claim:

1. A photonic based non-invasive surgery system comprising:
an imaging device for taking an image of a person's body to provide details of internal physiology;
at least two beam generators for generating beams of energy for delivery to a target in the person's body,
wherein the beams of energy intersect at a point;
means for feed-forward control for precalculating anticipated deflections and resulting pathways as the beams of energy travel throughout the person's body; and
means for feedback control through information gathered by the imaging device,
wherein the means for feed-forward control and the means for feedback control function in an integrated manner.

2. The system according to claim 1, wherein the imaging device includes a magnetic resonance imaging device or computed tomography device.

3. The system according to claim 2, wherein the imaging device includes a gantry table for moving the person in the magnetic resonance imaging device or computed tomography device.

4. The system according to claim 1, wherein the at least two beam generators generate the same type of energy.

5. The system according to claim 1, wherein the at least two beam generators generate different types of energy.

6. The radiotherapy system according to claim 5, wherein the beams of energy include radiation, ultrasound and microwave energy.

7. The system according to claim 1, wherein the target includes specific cells such as cancer cells or groups of cells including non-cancer cells.

8. The system according to claim 7, wherein the target includes lysosomes, mitochondria and other organelles in the cell.

9. The system according to claim 1, wherein the point is the target.

10. The system according to claim 1, wherein the means for feed-forward control includes a software program in a targeting computer for precalculating the target locations, anticipating deflections caused by the person's body's surface, bones and tendons, landmarks for the target acquisition, the target size, optimal beam sizes at the target, beam path working backward from the target, diffraction angles, deflection angles, beam diffusions, beam strengths needed at the target, absorption or attenuation rates along a pathway, power loss along a pathway, initial beam strengths required, beam size required at generator, number of beams needed, gantry robot position, robotic positioning of arms for each beam generator, anticipated movements in certain degrees of freedom, a person's movement range, a person's movement cycle, phase shifts and firing sequences.

12. A photonic based non-invasive surgery system comprising:
an imaging device for taking an image of a person's body to provide details of internal physiology;
at least two beam generators for generating beams of energy for delivery to a target in the person's body along a certain pathway,
wherein the beams of energy intersect at a certain point, and
wherein the beams of energy include different types of energy for delivery to the target along the certain pathway;
means for feed-forward control for precalculating anticipated deflections and the certain pathway as the beams of energy travel throughout the person's body; and
means for feedback control through information gathered by the imaging device.

13. The system according to claim 12, wherein the imaging device includes a magnetic resonance imaging device or computed tomography device.

14. The system according to claim 13, wherein the imaging device includes a gantry table for moving the person in the magnetic resonance imaging device or computed tomography device.

15. The system according to claim 12, wherein the at least two beam generators generate the same type of energy.

16. The system according to claim 12, wherein the at least two beam generators generate different types of energy.

17. The system according to claim 16, wherein the beams of energy include radiation, ultrasound and microwave energy.

18. The system according to claim 12, wherein the target includes specific cells such as cancer cells or groups of cells including non-cancer cells.

19. The system according to claim 18, wherein the target includes lysosomes, mitochondria and other organelles in the cell.

20. The system according to claim 12, wherein the point is the target.

21. The system according to claim 12, wherein the means for feed-forward control includes a software program in a targeting computer for precalculating the target locations, anticipated deflections caused by the person's body's surface, bones and tendons, landmarks for the target acquisition, the target size, optimal beam sizes at the target, beam path working backward from the target, diffraction angles, deflection angles, beam diffusions, beam strengths needed at the target, absorption or attenuation rates along a pathway, power loss along a pathway, initial beam strengths required, beam size required at generator, number of beams needed, gantry robot position, robotic positioning of arms for each beam generator, anticipated movements in certain degrees of freedom, person's movement range, person's movement cycle, phase shifts and firing sequences.

22. A photonic based non-invasive surgery system comprising:
an imaging device for taking an image of a person's body to provide details of internal physiology;
at least two beam generators for generating beams of energy for delivery to a target in the person's body,
wherein the beams of energy intersect at a certain point;
means for feed-forward control for precalculating anticipated deflections and resulting pathway as the beams of energy travel throughout the person's body;
means for feedback control through information gathered by the imaging device; and
a plurality of nanoparticles attached to the target or within the target.

23. The system according to claim 22, wherein the imaging device includes a magnetic resonance imaging device or computed tomography device.

24. The system according to claim 23, wherein the imaging device includes a gantry table for moving the person in the magnetic resonance imaging device or computed tomography device.

25. The system according to claim 22, wherein the at least two beam generators generate the same type of energy.

26. The system according to claim 22, wherein the at least two beam generators generate different types of energy.

27. The system according to claim 26, wherein the beams of energy include radiation, ultrasound and microwave energy.

28. The system according to claim 22, wherein the target includes specific cells such as cancer cells or groups of cells including non-cancer cells.

29. The system according to claim 28, wherein the target includes lysosomes, mitochondria and other organelles in the cell.

30. The system according to claim 22, wherein the point is the target.

31. The system according to claim 22, wherein the means for feed-forward control includes a software program in a targeting computer for precalculating the target locations, anticipated deflections caused by the person's body's surface, bones and tendons, landmarks for the target acquisition, the target size, optimal beam sizes at the target, beam path working backward from the target, diffraction angles, deflection angles, beam diffusions, beam strengths needed at the target, absorption or attenuation rates along a pathway, power loss along a pathway, initial beam strengths required, beam size required at generator, number of beams needed, gantry robot position, robotic positioning of arms for each beam generator, anticipated movements in certain degrees of freedom, person's movement range, person's movement cycle, phase shifts and firing sequences.

32. The system according to claim 22, wherein the nanoparticles include gold, carbon, iron, magnetic material, compound metal, tubes, balls, bubbles, springs, coils, rods and combinations thereof.

33. The system according to claim 22, wherein the means for feed-forward control and the means for feedback control function in an integrated or in an independent manner.

34. The system according to claim 22, wherein the nanoparticles are targeted to the target cells by an attached peptide, monoclonal antibody, monoclonal antibody fragment, or aptamer.

35. The system according to claim 22, wherein the nanoparticles are targeted to the mitochondria by an attached mitochondrial targeting peptide.

36. The system according to claim 31, wherein the pathway is defined with tolerances for automated adaptation for person specific applications.

37. A photonic based non-invasive surgery system comprising:
an imaging device for taking an image of a person's body to provide details of internal physiology;
at least one beam generator for generating beams of energy for delivery to a target in the person's body,
wherein at least one beam generator includes a beam processor for processing the beam from the beam generator, and
wherein the beams of energy intersect at a certain point;
means for feed-forward control for precalculating anticipated deflections and resulting pathway as the beams of energy travel throughout the person's body;
means for feedback control through information gathered by the imaging device; and
a plurality of nanoparticles attached to the target or within the target.
